SEQUENCE LISTING (10 sequences)

SEQ ID NO: 1 - DNA, 34 bp, Artificial Sequence
  IDH1 gene R132H mutation detection_Forward primer
agttgaaacaaatgtggaaatcaccaaatggcac

SEQ ID NO: 2 - DNA, 35 bp, Artificial Sequence
  IDH1 gene R132H mutation detection_Reverse primer
gccaacatgacttacttgatccccataagcattta

SEQ ID NO: 3 - DNA, 31 bp, Artificial Sequence
  IDH1 gene R132G mutation detection_Forward primer
agtggatgggtaaaacctatcatcataggag

SEQ ID NO: 4 - DNA, 32 bp, Artificial Sequence
  IDH1 gene R132G mutation detection_Reverse primer
accttgcttaatgggtgtagataccaaaagat

SEQ ID NO: 5 - DNA, 31 bp, Artificial Sequence
  IDH1 gene R132C mutation detection_Forward primer
agtggatgggtaaaacctatcatcatagtgt

SEQ ID NO: 6 - DNA, 32 bp, Artificial Sequence; identical to SEQ ID NO: 4
  IDH1 gene R132C mutation detection_Reverse primer

SEQ ID NO: 7 - DNA, 26 bp, Artificial Sequence
  IDH2 gene R172K mutation detection_Forward primer
gcctacctggtcgccatgggcgtaat

SEQ ID NO: 8 - DNA, 33 bp, Artificial Sequence
  IDH2 gene R172K mutation detection_Reverse primer
gatggcggctgcagtgggaccactattatctct

SEQ ID NO: 9 - DNA, 30 bp, Artificial Sequence
  IDH2 gene R172W mutation detection_Forward primer
gggtgaagaccattttgaaagtgccggccc

SEQ ID NO: 10 - DNA, 31 bp, Artificial Sequence
  IDH2 gene R172W mutation detection_Reverse primer
cctggctggaccaagcccatcaccattgaat

(19) United States
(12) Patent Application Publication
(10) Pub. No.: US 2023/0073129 A1
(43) Pub. Date: Mar. 9, 2023

(54) METHOD FOR INTRAOPERATIVELY OR POSTOPERATIVELY DETECTING IDH1 AND IDH2 GENE MUTATIONS IN GLIOMA TUMORS AND PRIMERS USED IN THIS METHOD

(71) Applicant: BAHCESEHIR UNIVERSITESI, Besiktas/istanbul (TR)

(72) Inventors: Timucin ASVAR, Kadikoy/tstanbul (TR); Melih ACAR, Kadikoy/tstanbul (TR); Alihan SURSAL, Kadikoy/istanbul (TR); Turker KILIC, Kadikoy/istanbul (TR)

(21) Appl. No.: 17/759,974

(22) PCT Filed: Apr. 9, 2021
(86) PCT No.: PCT/TR2021/050339

§ 371 Date: Aug. 2, 2022

(30) Foreign Application Priority Data
Apr. 10, 2020 (TR) 2020/05788

(51) Int. Cl. C12Q 1/6886

(52) U.S. Cl. CPC C12Q 1/6886; C12Q 2600/156; C12Q 1/6851

(57) ABSTRACT

The present invention relates to a method for intraoperatively or postoperatively detecting isocitrate dehydrogenase-1 (IDH1) and isocitrate dehydrogenase-2 (IDH2) gene mutations in glioma tumors and primers used in this method. In present invention, mismatched nucleotides were determined empirically, and the inventive primers were obtained by means of adapting the designs of the primers to detect the most common mutations in the IDH1 and IDH2 gene. The inventive primers comprise three 3′ terminal nucleotides and said nucleotides mismatch to wild-type DNA (WT-DNA). However, only two of them, the penultimate and adjacent nucleotide, indicate mismatch to mutant DNA. The inventive method and primers provide a gene mutation detection method with high sensitivity and specificity, that allows for obtaining fast and reliable results.

### TECHNICAL FIELD OF THE INVENTION

The present invention relates to a method for intraoperatively or postoperatively detecting isocitrate dehydrogenase-1 (IDH1) and isocitrate dehydrogenase-2 (IDH2) gene mutations in glioma tumors and to primers used in this method.

### STATE OF THE ART

Today, there are various mutation detecting methods used in the detection and differential diagnosis of brain tumors. In glioma tumors, detection of IDH1/2 mutations during the surgery, is a critical determinant on to what extent the tumor should be excised in surgery management or whether the post-operative radiotherapy and chemotherapy will be successful or not. The degree of surgical resection has a different recovery effect on patients who has the low-grade gliomas based on their molecular subtype. IDH mutant low-grade gliomas benefit from surgical resection more, therefore detecting IDH mutation intraoperatively is quite important.

In the state of the art, one of the conventional methods used primarily is the immunohistochemistry (IHC) method. IHC method is considered as most commonly used method for detecting the R132H mutations in the IDH1 gene after the surgical excision of glioma tumors. However, IHC-based techniques provide limited information about the IDH1 mutations. Because available antibodies can detect only one (R132H) mutation in the IDH1 gene, in other words, they cannot detect other mutations in the IDH1 or IDH2 (IDH1/2) genes. Moreover, since IHC is based on examining antibody-stained cells under microscope, this method is prone to misinterpretation as tumor cells with R123H mutation may go unnoticed in case these cells are quite rare in the tumor that is being analyzed.

As an alternative to IHC, DNA sequencing methods are commonly used to detect the mutations in IDH1/2 genes. Especially, Sanger sequencing is regarded as the golden standard for detecting IDH1/2 mutation, and commonly utilized by the centers which have the DNA sequencing substructure. However, if the mutation exists only in a small fraction of the cells generating the tumor, the sensitivity of Sanger sequencing may not detect a mutation present in the tumor. Both IHC and sequencing-based technique are not suitable for detecting IDH1/2 mutations in glioma tumors preoperatively and/or intraoperatively, due to the requirement of performing long-term assays during the surgery or in case biopsy samples are not available prior to the surgery. Therefore, a method for detecting mutations that is capable of intraoperatively detecting mutations is of great importance for the diagnosis and the treatment of glioma tumors and it should be a test that can yield results in a brief period of time, with the highest accuracy, sensitivity and specificity. This can only be possible by using genotyping methods that are based on real-time polymerase chain reaction (RT-PCR).

In the state of art, there are several genotyping methods that are suitable for detecting intraoperatively the IDH1/2 mutations for glioma tumors. Being one of the most preferred methods, polymerase chain reaction (PCR) genotyping using peptide nucleic acids (PNA) and/or locked nucleic acids (LNA) is also one of the most sensitive detection methods for the detection of the IDH1/2 genes mutation in Glioma tumors. In these techniques, PNA is used for inhibition of the wild type (WT) DNA amplification. LNA is used for specifically amplifying the mutant DNA sequence during PCR. In this manner, sensitivity and specificity of the test are increased [1].

In the state of art, another genotyping method is to use quencher fluorophore probes inherent to IDH1 mutations during PCR. In this method, the mutant DNA is detected through a strategy based on real-time monitoring of the DNA by using probes and selective amplification of DNA molecules carrying the IDH1 mutation during PCR [2].

Detecting mutation by using Allele-specific Amplification Refractor Mutation Systems (ARMS) PCR is another alternative genotyping method, wherein sequence specific primers are used specifically for replicating the target DNA in a mixture of target and non-target DNAs. Specificity of the reaction is ensured by selecting oligonucleotides. Primers are short single-stranded DNA molecules that are complementary structures to the bonding zone that will bind to the DNA to be amplified. This method requires several modifications within the primer sequences for specific replication. 3 ends (3′) of the primer that binds from the PCR primers to the point where the mutation occurs, are designed as mismatch with wild type DNA (wtDNA) while the terminal nucleotide is matched with the mutant tumor in conventional ARMS. However, said mismatch is not sufficient for PCR to distinguish mutant DNA and wild type DNA. Therefore, the penultimate nucleotide, which is the nucleotide preceding the nucleotide with the mutation is designed as a mismatch with both mutant and wild type DNA. This approach of primer design ensures that the wild type DNA is not replicated, while replicating the mutant DNA in the mixture. Because, in wild type DNA, there are 2 mismatched nucleotides at the end where the primer is bonded, and one mismatched nucleotide in mutant DNA. PCR cannot tolerate primers with two mismatched nucleotides; thus the sensitivity of PCR is improved [3].

The conventional methods (immunohistochemistry and DNA sequencing) in the state of art are not suitable for detecting the mutation of glioma tumors intraoperatively due to the time barrier and analytical steps of the methods. IDH1 or IDH2 mutation detection requires staining of its sections with antibodies and fixation of the tissue properly for IHC. The available antibody clone is limited to the single point mutation R132H, which is the most relevant mutation type of the IDH1 gene, and the R172K mutation of the IDH gene. However, the other rare mutations cannot be detected by using IHC techniques. Also, IHC is a complex multi-step method and requires several washing steps during overnight incubations and analytical steps. Therefore, IHC is not suitable for detecting gene mutation intraoperatively which requires a maximum result reporting time of one or two hours.

The Sanger sequencing method is the golden standard of gene mutation detection. However, since DNA sequencing requires many preliminary steps, it is not a suitable method for intraoperatively detecting gene mutation. In this method; DNA isolation from tumor tissue, specific amplification of the area containing the IDH1/2 gene on DNA with primers, purification of the amplified DNA, sequencing reaction, and capillary electrophoresis steps must be carried out sequentially and with high efficiency. In conclusion, the results must be lined up to a reference gene and thus, the presence of the mutation is detected. However, it is not possible to use the Sanger sequencing method for detecting gene mutations intraoperatively, due to the lack of instrumental setup in the operating room and the time limitation during surgery even though the Sanger sequencing method is the golden standard method for mutation detection.

Genotyping of IDH1/2 mutations by using modified primers such as peptide nucleic acid (PNA) and/or locked nucleic acid (LNA) is also the suitable and correct method, which can be used for intraoperative purposes. These methods feature high sensitivity and specificity, and kits, which are utilized in this method and commercialized for the detection of genetic mutation, are available in the state of the art. However, these methods have some limitations. The first of these limitations is that there are a limited number of global PNA and LNA producers around the world, and therefore PNA and LNA are relatively expensive chemical materials. This causes methods using PNA and LNA to be expensive. The second restriction originates from the fact that PNA and LNA are oligonucleotide-based molecules with higher affinity to DNA. They are conjugated to fluorophore molecules to be used in real-time PCR. However, the fact that the number of usable fluorophore conjugates and combinations thereof are limited imposes a further restriction on replication of mutations for the detection of combinations of PNA- and LNA-based probes.

Genotyping with quencher PCR probes is one of the alternative methods suitable for intraoperative approaches. However, these methods are relatively expensive just like the PNA- and LNA-based methods and the replication of different mutations is limited to the number of dye conjugates. In addition, it cannot be practiced in all kinds of mutations with high sensitivity and specificity, since probe sensitivities are not as high as PNA and LNA.

Conventional ARMS-based genotyping (design with 2 mismatching nucleotides) is one of the most suitable methods available in the state of art with regard to its simplicity, cost-efficiency and ease of use. However, there are problems in terms of sensitivity and specificity which are the most important features of a diagnostic test.

Consequently, limitations and insufficiencies of mutation detection methods available in the state of the art necessitated making and improvement in methods for detecting IDH1/2 gene mutations in glioma tumor.

### BRIEF DESCRIPTION AND OBJECTS OF THE INVENTION

In present invention, intraoperatively or postoperatively detecting IDH1 and IDH2 gene mutations in glioma tumors by an ARMS-derived method, and primers designed specifically for this method are described.

An object of the present invention is to ensure that intraoperative detection of IDH1 and IDH2 gene mutations in glioma tumors is performed rapidly and reliably. The inventive method and primers used therein allow for rapidly detecting IDH1/2 mutation during the surgical operation, thereby allowing for making decisions more efficiently regarding the procedure to be carried out on the tumor.

Another object of the present invention is to enable easy and rapid detection of several mutations simultaneously by using different fluorophore dyes and primer combinations.

Yet another object of the present invention is to provide an easily accessible and cost-efficient method of gene mutation detection. The inventive primers and chemical materials feature lower costs compared to the methods available in the state of the art since commonly used materials may be utilized in the present invention.

Yet another object of the present invention is to develop a method for detecting IDH1 and IDH2 gene mutation in a glioma, featuring much higher sensitivity and specificity compared to methods available in the state of the art.

Yet another object of the present invention is to ensure that gene mutation may be detected through selective mutant DNA amplification without the need for LNA, PNA and oligonucleotide-blocking nucleotide sequences.

### DESCRIPTION OF THE FIGURES

FIG. 1 illustrates the view of primer design approach in 3m-ARMS method ✓: match, [symbol] : mismatch, [symbol] : elongation, [symbol] : no elongation).

FIG. 2 illustrates the mutation-specific DNA replication in agarose gel in 3m-ARMS method.

FIG. 3 illustrates the overlapping of (e-h) Sanger sequencing results with 3m-ARMS method results, method with real-time PCR, and DNA replication performed by 3m-ARMS method in (a-d) real-time PCR.

FIG. 4 illustrates the amplification curves resulting from the sensitivity and specificity of PCR performed with 3m-ARMS primers.

FIG. 5 illustrates the comparison of the mutation detecting methods. (3m-ARMS (+) Sanger (+), Sanger (+) IHC (+) and 3m-ARMS (+) COLD-PCR & Sanger (+) columns indicate the consistent results, other columns indicate the inconsistent results. (+) indicates the detection of mutations and (−) indicates that there is no mutation detection.)

### DESCRIPTION OF ELEMENTS/SECTIONS/PARTS THAT CREATES THE INVENTION

Parts and sections are enumerated in order to provide a better understanding of the primers developed in the present invention, and names of parts and sections corresponding to each number are given below:
1. 3m-ARMS primer
2. Wild Type (WT) DNA
3. Mutant (MT) DNA

### DETAILED DESCRIPTION OF THE INVENTION

The present invention relates to a method for intraoperatively and postoperatively detecting IDH1 and IDH2 gene mutations, and primers designed specifically for this method.

The inventive primers have three mismatching nucleotides, which results in inhibition of primer elongation and DNA amplification. Thus, wild type DNA is not replicated, and mutant DNA replication and detection of mutations can be carried out in a sensitive manner. Owing to the inventive primers, which result in suitable primer elongation and DNA amplification, IDH R132H mutant DNA (MT-DNA) comprises 1 terminal match, 1 penultimate and 3 ante-penultimate mismatch nucleotides. Briefly, as illustrated in FIG. 1, while mutant DNA can be replicated, wild-type DNA is not, due to the fact that mutant DNA and 3m-ARMS primers include 2 mismatch, one matched nucleotide at the 3′ end of the primer, and wild type DNA on the other hand, include 3 mismatched nucleotides in the same place.

FIG. 1 illustrates how the design of the inventive primers is related to modified ARMS method. The primer designed for wild type DNA has 3 overlapping mismatch nucleotides at the 3′ terminal end, while the 2 previous nucleotides are designed to as mismatches, the last base for the mutant DNA is designed to be a match. Mismatch nucleotides are added to the 3′ terminal end of the primer. Thus, while wild type DNA cannot be replicated with polymerase, mutant DNA can be replicated, and gene mutation detection can be performed rapidly and in a sensitive manner.

The primer design (3m-ARMS primer) comprising three mismatch nucleotides increases the specificity of gene mutation detection. In the present invention, adding a third mismatch nucleotide to conventional ARMS primer design not only increases the accuracy of detection of mutations but also allows for performing mutation detection much faster compared to the methods available in the state of the art. Detection of IDH2 R172K with IDH1 R132H, R132G and R132C gene mutations is performed intraoperatively and postoperatively by means of the inventive 3m-ARMS primers.

Normally, 3 ends (3′) of terminal nucleotide of one of the PCR primers in conventional ARMS are designed to match mutant tumor DNA and mismatch to wild type DNA (wtDNA). The penultimate nucleotide is designed such that it is a mismatch to both mutant DNA and WT-DNA along with the terminal mismatch nucleotide. The selection of the penultimate nucleotide is based on the nucleotide with terminal mismatch, and the penultimate nucleotide ensures the DNA amplification of the mutant DNA, however, two nucleotide mismatches for WT-DNA cannot tolerate the wtDNA amplification. In PCRs utilizing conventional ARMS, it is observed that having only two mismatching nucleotides does not always provide specific amplification of mutant DNA, since sometimes WT-DNA is also replicated. Thus, sensitivity of gene mutation detection is improved. Therefore, the inventive primers are designed by adding a third additional mismatch nucleotide. Accordingly, the inventive method for detecting gene mutation can be called as 3′3 mismatch ARMS or 3m-ARMS method. Basically, in the inventive 3m-ARMS method, three 3′ terminal nucleotides in the PCR primers on the DNA chain subjected to mutation indicate mismatch to WT-DNA, however, only two of these, the penultimate and adjacent nucleotides, are mismatches with the mutant DNA. In the present invention, mismatch nucleotides have been determined empirically, and the inventive primers have been obtained by adapting the design of the primers to detect most common mutations in the IDH1 and IDH2 gene (Table 1). The inventive 3m-ARMS primers are designed to have two primers for each mutation, as forward (F) and reverse (R), have the SEQ. ID 1-10 nucleotide sequences.

TABLE 1
Information of designed primers

| Mutation detected | | |
|---|---|---|
| by the primer | Primer name | Primer sequence |
| IDH1 gene R132H | Primer 1_F | 5′-AGTTGAAACAAATGTGGAAATCACCAAATGGCAC-3′ |
| primers | | |
| mutation detection | Primer 1_R | 5′-GCCAACATGACTTACTTGATCCCCATAAGCATtta-3′ |
| IDH1 gene R132G | Primer 2_F | 5′-AGTGGATGGGTAAAACCTATCATCATAGgag-3′ |
| primers | | |
| mutation detection | Primer 2_R | 5′-ACCTTGCTTAATGGGTGTAGATACCAAAAGAT-3′ |
| IDH1 gene R132C | Primer 3_F | 5′-AGTGGATGGGTAAAACCTATCATCATAGtgt-3′ |
| primers | | |
| mutation detection | Primer 3_R | 5′-ACCTTGCTTAATGGGTGTAGATACCAAAAGAT-3′ |
| IDH2 gene R172K | Primer 4_F | 5′-GCCTACCTGGTCGCCATGGGCGTaat-3′ |
| primers | | |
| mutation detection | Primer 4_R | 5′-GATGGCGGCTGCAGTGGGACCACTATTATCTCT-3′ |
| IDH2 gene R172W | Primer 5_F | 5′-GGGTGAAGACCATTTTGAAAGTGCCGGCCC-3′ |
| primers | | |
| mutation detection | Primer 5_R | 5′-CCTGGCTGGACCAAGCCCATCACCATTGaat-3′ |

Nucleotides present in lowercase letters indicate mismatching nucleotides.

The 3m-ARMS real-time PCR analysis conditions in which the inventive gene mutation detecting method is applied are as follows: 95° C. for 30 seconds, PCR for 45 cycles; degradation of DNA for 30 seconds at 95° C., binding of the inventive primers for 20 seconds at 62° C., and elongation of DNA polymerase for 10 seconds at 72° C., final polymerase elongation at 72° C. for 2 minutes. Melting curve is obtained under 95° C. for 10 seconds, 60° C. for 60 seconds and 97° C. for 1 second.

The neurosurgeon resects the tumor fragments, during the surgical operation. In other words, these tumor fragments are resected from the patient's body during surgery. This is a standard surgical operation. These tumor fragments are then transmitted to the laboratories for analysis. The present invention gets involved after this point and it does not comprise a surgical implementation. In the present invention, a method that provides much more economic, fast and reliable results has been developed for the analysis of fragments of tumor resected from the patient's body during surgery. The present invention comprises only the analysis phase.

Intraoperatively Detecting Mutation with the Method for Detecting Mutation Gene and Inventive Primers:

The neurosurgeon resects the tumor fragments during the surgical operation of glioma tumors and sends them to the laboratory for tissue analysis. In the present invention, at least 5-25 mg of tumor tissue is sufficient for mutation detection, and the tumor tissue is primarily subjected to alkaline lysis DNA extraction method by means of alkaline lysis DNA extraction solutions. Tumor is cut into small pieces, and 200 μl of solution A (alkaline buffer, 1 mM of Na2EDTA, 25 mM of NaOH, pH: 12) is added onto it, and incubated at 95° C. for 8 minutes. Then, solution B (neutralization buffer, 40 mM of Tris-HCl buffer) is added to said mixture, and centrifuged immediately after, subsequently, the supernatant is separated to be tested with the inventive 3m-ARMS method for detecting gene mutation. The inventive 3m-ARMS method has been optimized for real-time PCR device. Thus, the inventive method is adapted to obtain results within approximately 1 hour during the surgery. The inventive 3m-ARMS primers and the PCR conditions thereof specific to optimized PrePCR mastermix solution (1 Unit Taq DNA Polymerase, 2 mM of dNTPs, 25 mMMg of Cl2, 15 mM of KCl and 0.1% DMSO) are stated above. Positive samples begin being amplified around the 20^{th} cycle of the PCR, and last approximately 35 minutes. Completion of the total PCR reaction lasts approximately 60 minutes. Mutation results for specific IDH mutations for each designed primer can be read by utilizing amplification curves and CT values.

Postoperatively Detecting Mutation with the Inventive Method for Detecting Mutation Gene and Primers:

The process steps described above in intraoperative mutation detection method can also be implemented to sectioned formalin fixed paraffin embedded (FFPE) tissue and to tissue samples obtained after surgery. The same process steps apply. In addition, the accuracy of the test was tested on FFPE samples.

Method for intraoperatively or postoperatively detecting isocitrate dehydrogenase-1 (IDH1) and isocitrate dehydrogenase-2 (IDH2) gene mutations in glioma tumors comprises the process steps of;
Subjecting 5-25 mg of tumor tissue to alkaline lysis DNA extraction method by means of alkaline lysis DNA solutions,
Cutting the tumor into small pieces and adding a solution which has a pH of 12 and contains alkali buffer, 1 mM of Na2EDTA, 25 mM of NaOH into it and incubating at 95° C. for 8 minutes.
Adding neutralization buffer containing 40 mM of Tris-HCl buffer solution into said mixture and performing short centrifugation immediately after,
Performing real-time PCR reaction of the tumor tissue supernatant obtained by centrifugation with 45 cycles at 95° C. for 30 seconds in PrePCR mastermix solution that contains real-time PCR 1 Unit Taq DNA Polymerase, 2 mM of dNTPs, 25 mMMg of Cl2, 15 mM of KCl and % 0.1 DMSO, separating DNA at 95° C. for 30 seconds, binding SEQ. ID 1-10 primer pairs at 62° C. for 20 seconds, and elongating DNA polymerase at 72° C. for 10 seconds, and elongating last polymerase for 2 minutes at 72° C.,
Determining the presence of the gene mutation by utilizing amplification curves and CT values generated from real-time PCR data.

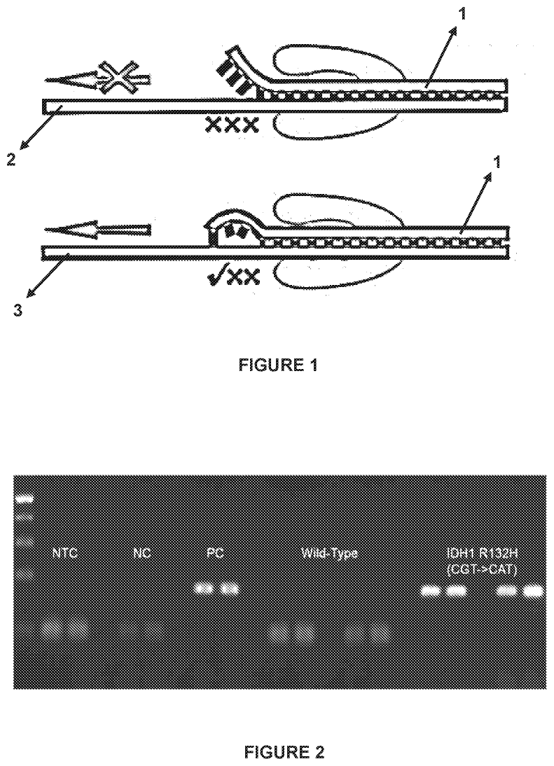

In the present invention, agarose gel analysis of PCR with 3m-ARMS primers has been performed in order to detect G395A (R132H) in tumor samples and it is illustrated in FIG. 2. In said analysis, standard DNA bands were loaded into the first well on the left and every sample was subjected to electrophoresis by means of loading into the gel in double wells adjacent to each other. Here, one single well remained empty among different samples. Two tumor samples carrying only G395A mutation (marked as IDH1 R132H in image) and sample of positive control (PC) gave a specific PCR band on said gel. The smaller faint bands are the primary dimers. As seen in FIG. 2; while DNA can be replicated in PCR that is performed with positive control and IDH1 R132H mutation in agarose gel image on the left side, there has not been a replication in negative control and wild-type DNAs.

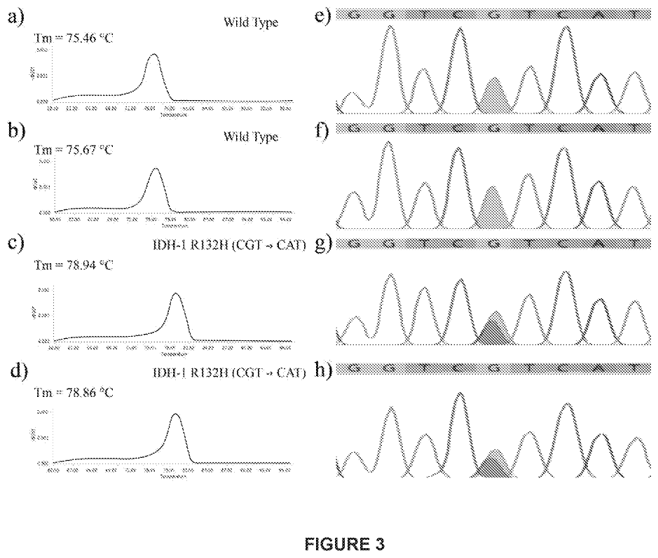

Mutation detection at real-time PCR is performed via melting temperature (TM) analysis. These melt at different temperatures since the nucleotide contents of mutant DNA and wild DNA are different (FIG. 3 a-d). It indicates that wild type samples (c and d) and mutant samples (e and f) can be separated from each other in terms of melting temperature (TM) at the end of the qRT-PCR based 3m-ARMS analysis. Only mutant samples give a distinctive peak with a high Tm (78-79° C.). Wild type DNA, on the other hand, can be easily distinguished as they indicate melting curve at a lower temperature (75-76° C.). The accuracy of the results was also indicated by Sanger sequencing. Sanger sequencing results analyzed on position 395 of IDH1 gene and on the gel (a) showing the nucleotide in the periphery thereof were shown for wild type (e and f) and mutant samples. Wild type samples show only one peak for nucleotide G at position 395, whereas mutant samples show two peaks for G and A nucleotides at the same position. Colored peaks indicate points where mutations and wild-type nucleotides occur. Results observed in FIG. 3 has importance for indicating that inventive gene mutation detecting method operates intraoperatively.

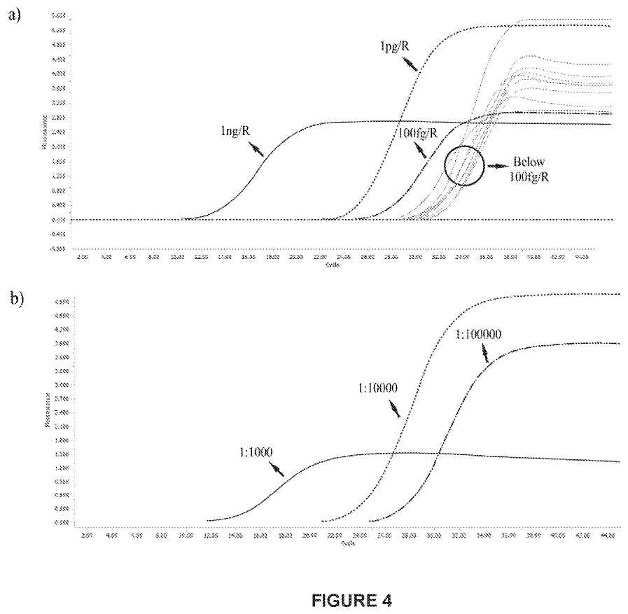

FIG. 4 illustrates the sensitivity and specificity characteristics of the PCR performed with the inventive 3m-ARMS primers. Minimum DNA concentration in which the mutant test functions were determined in order to test the sensitivity in the PCR assays carried out with designed primers. Rising curves indicate that DNAs of different concentrations begin rising at different PCR cycles. The designed test can even operate with 100 femtograms of DNA per reaction (100 fg/R). The specificity of the developed test has been illustrated in the graphic below. The ascending graphics indicate replicating DNAs. Here, it was aimed to measure the sensitivity of the test by mixing the mutant DNA and wild type DNA at different rates. As is observed, even 1 mutant DNA in 100,000 wild-type DNA can be specifically replicated. The amplification curves illustrated in FIG. 4 indicate the ct (amplification threshold cycle) values of each DNA sample. Arrows in FIG. 4-a indicate the degrees of DNA content ranging from 100 fg/R per reaction to 1 nanogram/R per reaction. Wild-type and mutant DNAs were tested separately in said analysis. Here, it is observed that the inventive method for detecting mutation is capable of PCR amplification and mutation detection even though at a concentration of 100 fg/R. In FIG. 4-b, arrows illustrate that ratios of mutant DNA/WT-DNA mixtures ranging from 1:1000 to 1:100,000. As illustrated in the figure, the higher concentration and higher relative ratio of mutant DNA provide lower ct values for specific amplification of the mutant DNA. Moreover, mutant DNA can be replicated specifically even in the case of mixing 100,000 wild-type DNA with a mutant DNA.

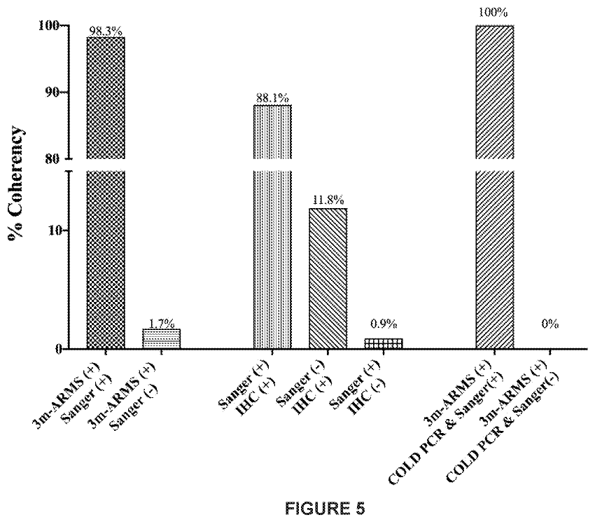

In the present invention, consistency of correct results between methods was ensured based on the Sanger sequencing method, which is considered as the gold standard method. The consistency indicates matching results for the two techniques that are being compared. As a result of the comparison, method for detecting gene mutation with the inventive 3m-ARMS primers indicates 98.3% coherency with the Sanger sequencing method, while it is 88.1% coherent with the IHC method. However, it was concluded that even the Sanger sequencing, which is considered as the golden standard, may be in accurate in accordance with the comparison made with Sanger sequencing method after the COLD-PCR. In FIG. 5, it is observed that inventive gene mutation detecting method utilizing 3m-ARMS primers provides more consistent results than IHC. Moreover, the fact that the inventive gene mutation detection method yields more consistent results on its own than the Sanger method was shown with Sanger sequencing results performed with COLD-PCR.

The inventive method for detecting mutation can be used in;
Differential diagnosis of glioma tumors based on IDH1 and IDH2 mutations,
Intraoperative detection of IDH1 and IDH2 mutations in Glioma tumors,
Detection of IDH1 and IDH2 mutations in acute myeloid leukemia (AML),
Postoperative detection of IDH1 and IDH2 mutations.

### REFERENCES

[1] Kanamori, M., Kikuchi, A., Watanabe, M., Shibahara, I., Saito, R., Yamashita, Y., Sonoda, Y., Kumabe, T., Kure, S., Tominaga, T. (2014), Rapid and sensitive intraoperative detection of mutations in the isocitrate dehydrogenase 1 and 2 genes during surgery for glioma, Journal of neurosurgery, Journal of neurosurgery, 120(6), 1288-1297.

[2] Ohka, F., Yamamichi, A., Kurimoto, M., Motomura, K., Tanahashi, K., Suzuki, H., Aoki, K., Deguchi, S., Chalise, L., Hirano, M., Kato, A., Nishimura, Y., Hara, M., Kato, Y., Wakabayashi, T., Natsume, A. (2017), A novel all-in-one intraoperative genotyping system for IDH1-mutant glioma, Brain tumor pathology, 34 (2), 91-97.

[3] Catteau, A., Girardi, H., Monville, F., Poggionovo, C., Carpentier, S., Frayssinet, V., Voss, J., Jenkins, R., Boisselier, B., Mokhtari, K., Sanson, M., Peyro-Saint-Paul, H., Giannini, C. (2014), A new sensitive PCR assay for one-step detection of 12 IDH1/2 mutations in glioma, Acta neuropathologica communications, 2(1), 58

1. A primer pair for intraoperatively or postoperatively detecting isocitrate dehydrogenase-1 (IDH1) or isocitrate dehydrogenase-2 (IDH2) gene mutations in glioma tumors, characterized in that; one of the forward-reverse primer pairs having SEQ. ID 1-2, SEQ. ID 3-4, SEQ. ID 5-6, SEQ. ID 7-8, or SEQ. ID 9-10 nucleotide sequences comprises three overlapped mismatched nucleotides at the 3′ terminal end thereof for the wild type DNA (WT-DNA) and the last one of these nucleotides are the matching nucleotide for the mutant DNA and two nucleotides from the last one are the mismatching nucleotides for the mutant DNA.

2. A primer pair according to claim 1, characterized in that; in the primer pair used for detecting IDH1 gene R132H mutation, the forward primer has the SEQ. ID 1 nucleotide sequence, and the reverse primer has the SEQ. ID 2 nucleotide sequence.

3. A primer pair according to claim 1, characterized in that; in the primer pair used for detecting IDH1 gene R132G mutation, the forward primer has SEQ. ID 3 nucleotide sequence, and the reverse primer has the SEQ. ID 4 nucleotide sequence.

4. A primer pair according to claim 1, characterized in that; in the primer pair, used for detecting IDH1 gene R132C mutation, the forward primer has SEQ. ID 5 nucleotide sequence, and the reverse primer has the SEQ. ID 6 nucleotide sequence.

5. A primer pair according to claim 1 characterized in that; in the primer pair, used for detecting IDH2 gene R172K mutation, the forward primer has SEQ. ID 7 nucleotide sequence, and the reverse primer has the SEQ. ID 8 nucleotide sequence.

6. A primer pair according to claim 1, characterized in that; in the primer pair, used for detecting IDH2 gene R172W mutation, the forward primer has SEQ. ID 9 nucleotide sequence, and the reverse primer has the SEQ. ID 10 nucleotide sequence.

7. A method for intraoperatively or postoperatively detecting isocitrate dehydrogenase-1 (IDH1) and isocitrate dehydrogenase-2 (IDH2) gene mutations in glioma tumors characterized in that, it comprises the process steps of;
Subjecting 5-25 mg of tumor tissue to alkaline lysis DNA extraction method by means of alkaline lysis DNA solutions,
Cutting the tumor into small pieces and adding a solution which has a pH of 12 and contains alkali buffer, 1 mM of Na₂EDTA, 25 mM of NaOH into it and incubating at 95° C. for 8 minutes.
Adding neutralization buffer, containing 40 mM of Tris-HCl buffer solution into said mixture and performing short centrifugation immediately after,
Performing real-time PCR reaction of the tumor tissue supernatant obtained by centrifugation with 45 cycles at 95° C. for 30 seconds in PrePCR mastermix solution that contains real-time PCR 1 Unit Taq DNA Polymerase, 2 mM of dNTPs, 25 mMMg of Cl2, 15 mM of KCl and 0.1% of DMSO, separating DNA at 95° C. for 30 seconds, binding SEQ. ID 1-10 primer pairs at 62° C. for 20 seconds, and elongating DNA polymerase at 72° C. for 10 seconds, and elongating last polymerase for 2 minutes at 72° C.,
Determining the presence of the gene mutation by utilizing amplification curves and CT values generated from real-time PCR data.

8. A method according to claim 7, characterized in that; the gene mutations are IDH1 gene R132H mutation, IDH1 gene R132G mutation, IDH1 gene R132C mutation, IDH2 gene R172K mutation and/or IDH2 gene R172W mutation.
